(19) United States
(12) Patent Application Publication
(10) Pub. No.: US 2006/0052298 A1
(43) Pub. Date: Mar. 9, 2006

(54) Use of stable glutamine derivatives to improve drug absorption

(76) Inventors: Richard L Guerrant, Charlottesville, VA (US); Nathan M Thielman, Durham, NC (US); Gerly Anne de Castro Brito, Fortaleza (BR); Aldo A M Lima, Fortaleza (BR)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/530,805

(22) PCT Filed: Oct. 10, 2003
(86) PCT No.: PCT/US03/32379

§ 371 Date: Apr. 8, 2005

Related U.S. Application Data
(60) Provisional application No. 60/418,008, filed on Oct. 11, 2002.

(51) Int. Cl. A61K 38/10; A61K 38/16

(52) U.S. Cl. 514/12

(57) ABSTRACT

The present invention relates to compositions and their use to enhance the uptake of pharmaceutical agents administered to mammalian species, including humans. More particularly, the present invention is directed to the administration of glutamine and stabilized glutamine derivatives to enhance the uptake of orally administered therapeutic agents.

### RELATED APPLICATIONS

This application claims priority under 35 USC § 19(e) to U.S. Provisional Application Ser. No. 60/418,008, filed Oct. 11, 2002, the disclosure of which is incorporated herein by reference.

### US GOVERNMENT RIGHTS

This invention was made with United States Government support under Grant No. 5 U01 AI 26512-14, awarded by National Institutes of Health. The United States Government has certain rights in the invention.

### BACKGROUND

The mucosal surface epithelial of the intestine represents an important barrier between deleterious agents within the intestinal lumen and the organism. Furthermore, the intestinal surface also functions as the primary tissue for absorbing nutrients and orally administered pharmaceuticals. Damage to the intestinal epithelium can interfere with both the barrier function as well as the absorptive function of the intestine. This leads to the result that some compounds are more readily absorbed by a disrupted gut (for example lactulose absorption increases) whereas some compounds are absorbed to a lesser extent by a disrupted gut (for example mannitol absorption decreases).

It is anticipated that damage to the intestinal epithelium may adversely impact the absorption of certain orally administered drugs resulting in a decreased efficacy of such treatments. For example, intestinal infections and HIV enteropathy have been found to cause malabsorption (histologically manifested by villus blunting and atrophy) that leads to a progressive deterioration in nutritional status. Persistent diarrhea and wasting are predominant manifestations of HIV infection in developing areas, and individuals with AIDS, that exhibit chronic diarrhea and wasting have a worse prognosis than cohorts living in the same conditions with the same CD4 counts but without diarrhea. In general, malabsorption due to suboptimal functioning of the alimentary system is believed to decrease a wide variety of therapeutic regiments administered orally to patients. Accordingly there is a need to treat malabsoption issues in conjunction with orally administered therapies. This is particularly relevant when treating HIV positive and AIDs patients with orally administered antiviral therapies.

Glutamine (Gln) is the primary fuel for both enterocyte and the colonocyte and is necessary for the maintenance of intestinal structure in both normal and stressed states (Cario et al., Eur J Clin Invest. 2000 May 30(5):419429). Studies have shown glutamine supplementation to prevent villous atrophy, and bacterial translocation, conditions associated with standard parenteral nutrition (Van Der Hulst et al., Lancet 1993 341:1363). Glutamine plays a pivotal role in several metabolic pathways. Its importance in tissue culture has long been recognized, and it is a key nitrogen donor for the biosynthesis of nucleotides, amino sugars, and amino acids in mammalian cells. It is anticipated that the availability of glutamine will be especially important during persistent diarrhea and malnutrition, when the mucosal barrier function is often disrupted. Animal studies have shown that glutamine-enriched nutrition can attenuate bacterial translocation, improve nutritional status, decrease intestinal injury and result in improved survival in a lethal model of methotrexate-induced enterocolitis.

Alanyl-glutamine (Ala-Gln) is a stable glutamine derivative that has been shown to be much more stable in acidic water solutions (such as they would be expected to face in a patient's stomach or intestine) and to drive salt and water absorption comparable to, if not better, than glucose (see U.S. Pat. No. 5,561,111, the disclosure of which is expressly incorporated herein). Stable glutamine derivatives are useful not only in malnourished children with diarrhea, but also in patients kept too long on parenteral (IV) fluids or tube feedings or in those with damaged intestinal mucosa from infection or chemotherapy.

The present invention is directed to the use of glutamine, and stable glutamine derivatives thereof, in combination with standard therapeutic drugs, to improve the absorbance of the drugs from the intestinal lumen and thus enhance the effectiveness of the therapeutic agent. In accordance with one embodiment a therapeutic regiment is provided comprising the administration of both glutamine (or a glutamine derivative) and a therapeutic agent to a patient, wherein the patient is suffering from a damaged intestinal mucosa, to stimulate absorption of the therapeutic agent and maintain the integrity of the intestinal mucosa

### SUMMARY OF VARIOUS EMBODIMENTS OF THE INVENTION

The present invention is directed to compositions comprising glutamine rich peptides and a pharmaceutical agent. Furthermore, the glutamine compositions of the present invention provide a novel approach to provide oral or parenteral adjunctive therapy to improve drug absorption in humans or animals with damaged intestinal mucosa Such damage may arise from conditions such as AIDS, enteric infections and the administration of chemotherapy.

### BRIEF DESCRIPTION OF THE DRAWINGS

FIG. 1: Participant Flow in the Study of Diarrhea, Antiretroviral Drug Malabsorption in AIDS Patients with Wasting and Improvements with Glutamine or Alanyl-glutamine.

FIG. 2: Clinical Responses with Glycine, Glutamine, Low and High Dose Alanyl-glutamine for 7 days by Diarrhea Frequency and Consistency.

FIG. 3a: Glutamine and Alanyl-Glutamine Improve NRTI Absorption in AIDS patients with Diarrhea and Wasting. For all treatment groups including NNRTI and PI; 12 of 19 (63%) drug levels improved on high dose Ala-Gln (p=0.02), 12 of 21 (57%) drug levels improved on Gln (p=0.03) and 15 of 30 (50%, ns) of levels improved on low dose Ala-Gln; vs. 4 of 20 (20%) on glycine.

FIG. 3b: Percent Improvement of Paired Antiretroviral Drug Levels by Treatment Groups.

FIG. 4: Percent Improvement of Mannitol Excretion in AIDS Patients with Diarrhea and Wasting (from mean of 4.73±3.18 for 26 pre-study values).

### DETAILED DESCRIPTION OF EMBODIMENTS

Definitions

In describing and claiming the invention, the following terminology will be used in accordance with the definitions set forth below.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “pharmaceutical agent” relates to any therapeutic compound that is administered to treat an individual for a disease or malady.

As used herein, the term “treating” includes prophylaxis of a specific disease or condition, or alleviation of the symptoms associated with a specific disease or condition and/or preventing or eliminating said symptoms.

As used herein, the term “intestinal damage” relates to lesions to the mucosa and/or epithelial cells of the intestine that impact the barrier and absorption capabilities of the intestine. Similarly the term “compromised intestinal function” refers to a condition wherein a mammal has a decrease capacity, relative to the population as a whole, to absorb pharmaceutical agents from the lumen of the digestive tract.

As used herein, the term “stabilized derivative of glutamine” relates to derivatives of glutamine that are more resistant to degradation than glutamine, when those compounds are exposed to elevated temperatures or acidic solutions.

As used herein, the term “glutamine-bearing compound” relates to compounds that comprise a glutamine residue and includes glutamine itself as well as polyglutamine and stabilized derivatives of glutamine.

As used herein, the term “secondary peptide” or “secondary protein” relate to peptides and proteins that have not been modified to contain glutamine rich regions. Typically, these peptides and proteins represent native protein sequences or fragments thereof. However, the term also includes synthetic amino acid sequences.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is lie or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

The term “pharmaceutically-acceptable salt” refers to salts which retain the biological effectiveness and properties of the parent compound and which are not biologically or otherwise undesirable. In many cases, the glutamine-bearing compounds of the present invention are capable of forming acid and/or base salts by virtue of the presence of amino and/or carboxyl groups, or groups similar thereto.

### Embodiments

The importance of glutamine's role in cell proliferation has been previously implicated by the fact that glutamine and its derivatives are essential for nucleic acid biosynthesis and are key intermediates for the cell at the time of replication. Despite the impressive potential of glutamine in enhancing rehydration and intestinal repair, it is potentially unstable, especially in acidic solutions (such as the stomach). Conversely, stable glutamine derivatives are capable of surviving the digestive system of patients and deliver sufficient amounts of glutamine to the patient to treat conditions associated with dehydration, malnutrition, or tissue injury (see U.S. Pat. No. 5,561,111, the disclosure of which is expressly incorporated herein). In addition, stable glutamine derivatives provide the solubility that free-form glutamine lacks and the glutamine derivatives can be cooked, pasteurized and sterilized, as well.

Applicants have now discovered that the administration of glutamine or stabilized glutamine derivatives can enhance the uptake of orally administered pharmaceutical agents, particularly for those individuals that suffer from intestinal damage. Accordingly, the present invention is directed to compositions and methods of improving the efficacy of orally administered pharmaceuticals, by enhancing the uptake of orally administered drugs by the intestinal cells.

In accordance with one embodiment of the present invention a method of enhancing the absorption of a pharmaceutical agent administered orally to a mammal is provided. The method comprises the steps of administering a composition comprising a glutamine-bearing compound in conjunction with the administration of the pharmaceutical agent. In one embodiment the glutamine composition is administered prior to the administration of the pharmaceutical agent, however in alternative embodiments the glutamine composition can be administered after or simultaneously with the administration of the pharmaceutical agent. In another embodiment the patient is first administered a composition comprising a glutamine-bearing compound for one to seven days prior to the administration of the pharmaceutical agent. The patent then continues to receive the glutamine composition while simultaneously receiving the pharmaceutical agent. In one embodiment the pharmaceutical agent is combined with the glutamine-bearing compound to form a single pharmaceutical composition.

The glutamine composition may comprise glutamine itself, a polymer of glutamine or a stabilized derivative of glutamine. Glutamine can be stabilized by coupling it with one or more additional amino acids, coupling glutamine with glucose, coupling glutamine with glucose and one or more additional amino acids, or acylating glutamine with a C₂-C₆ carboxylic acid. Such modified compounds are capable of surviving the digestive system of patients while at the same time delivering sufficient amounts of glutamine to the patient to obtain effective amounts in the patient's system. These compositions treat damage to intestinal tissues by enhancing the first step of intestinal epithelial repair, named restitution.

In one embodiment of the present invention, a composition is provided comprising a peptide that is enriched for glutamine residues. The peptide can be either a native peptide or a recombinant protein that is engineered to be enriched for glutamine residues. Furthermore, the glutamine bearing peptides of the present invention may also include internal protease cleavage sites that release the GLN or ALA-GLN repeat region upon contact with the protease. Suitable protease cleavage sites and methods of producing recombinant proteins containing such site are well know to those skilled in the art. Suitable protease cleavage sites include but are not limited to trypsin, chemotrypsin, Factor Xa and TEV.

In one embodiment the glutamine enriched protein comprises an amino acid sequence selected from the group consisting of (GLN)ₙ, (ALA-GLN)ₙ, (GLN-Y-X)ₙ, (ALA-GLN-Y)ₙ, (ALA-GLN-Y)ₙ-[protease cleavage site]-(ALA-GLN-Y)ₚ, (GLN-Y-X)ₙ-[protease cleavage site]-(GLN-Y-X)ₚ and {(ALA-GLN)ₙ-[protease cleavage site]-(ALA-GLN)ₚ}ₘ wherein X and Y are independently GLN or ALA, n and p are integers independently selected from a range of 1 to 100, and m is an integer ranging from 1 to 20. In one embodiment n and p are integers independently selected from a range of 1 to 50 or from 1 to 20. In accordance with one embodiment a glutamine-bearing compound comprises an amino acid sequence of the general formula ALA-(GLN)ₙ, (ALA-GLN)ₙ or [(ALA-GLN)ₙ-protease cleavage site-(ALA-GLN)ₚ]ₘ wherein n and p are integers independently selected from a range of 1 to 5, and m is an integer ranging from 1 to 20, or in another embodiment n and p are integers independently selected from a range of 1 to 3 and m is an integer ranging from 1 to 5.

In one embodiment the glutamine-bearing compound is a peptide ranging from 2 to about 20 amino acids in length and comprise one or more sequences selected from the group consisting of (ALA-GLN)ₙ, ALA-(GLN)ₙ, MET(ALA-GLN)ₙ or MET[(ALA-GLN)_{q}-protease cleavage site-(ALA-GLN)ₚ]ₘ wherein n is 1-20, p and q are integers independently selected from the range of 1-3 and m is an integer ranging from 1 to 3. In one embodiment the number of total amino acid groups present in the compounds used in the present method ranges from 2 to 5 (formed from coupling 1 to 4 amino acids with glutamine). Preferably, the compound is a dipeptide or tripeptide. In one embodiment the compound is selected from the group consisting of alanyl-glutamine, alanyl-glutaminyl glutamine and gamma-glutamyl glutamine. In accordance with one embodiment the glutamine-bearing compound is a peptide comprising the amino acid sequence (ALA)_{W}(GLN)_{X}(ALA)_{y}(GLN)_{z} wherein w, x, y and z are independently 1-5. The compounds used in the present invention are known and can be prepared using conventional peptide coupling reactions, such as on a solid phase peptide synthesizer or using 1,3-diisopropyl-carbodiimide (DIPCDI) activation in solution coupling, as described in Hudson, J. Org. Chem., 53(3):617-624 (1988) and Bodansky et al, Synthesis, pp. 453-463 (1991).

In accordance with one embodiment a patient suffering from intestinal damage is administered a composition comprising a glutamine-bearing compound to enhance the uptake of an orally administered therapeutic drug. The intestinal damage may be unrelated to the condition intended to be treated by the therapeutic agent. For example the intestinal damage may be the result of chemotherapy or radiation treatments wherein the therapeutic agent is an orally administered anticancer agent. Alternatively, the intestinal damage may be a direct result or symptomatic of the condition/disease to be treated with the therapeutic agent. For example, persistent diarrhea and wasting are predominant manifestations of HIV infection, especially in developing areas.

Changes in intestinal function have been well documented in AIDS. For example, lactulose:mannitol ratios in urine (a widely used measure of malabsorption and intestinal permeability) in HIV-infected patients both with and without diarrhea have striking differences from controls, suggesting a decrease in the functional absorptive surface of the intestine (Tepper et al., 1994; Silva et al., 1997). Lactulose is typically not significantly absorbed by a healthy whereas mannitol is absorbed by a healthy gut. As the gut is disrupted lactulose uptake increase and mannitol uptake decreases. This functional disruption parallels the physical alteration of the mucosa described by microscopy, and correlates with the intensity of opportunistic infection (e.g. cryptosporidiosis). Both intestinal infections and HIV enteropathy have been found to cause malabsorption that is manifested histologically by villus blunting and atrophy and leads to progressive deterioration in nutritional status. It is believed that both opportunistic pathogens and the HIV itself may contribute to diarrhea and the AIDS-wasting syndrome. Individuals with AIDS, chronic diarrhea and wasting have a worse prognosis than cohorts living in the same conditions with the same CD4 counts but without diarrhea. Furthermore, reduced plasma levels of anti mycobacterial agents have been documented in patients coinfected with HIV, including those receiving directly observed therapy (Sahai et al., 1997; Beming et al., 1992; Patel et al., 1995; Gordon et al., 1994, Peloquin et al., 1996, and Taylor and Smith, 1998). Most of these authors suggest that malabsorption may be the underlying cause, presumably secondary to the process of mucosal disruption outlined above.

The glutamine-bearing compounds of the present invention can be used in conjunction with antiretroviral drugs to improve the uptake of such drugs in patients suffering from a retroviral infection. In accordance with one embodiment of the present invention a method for treating HIV positive patients is provided. The method comprises the steps of administering a composition comprising a glutamine-bearing compound in conjunction with standard antiretroviral drugs, including protease inhibitors and reverse transcriptase inhibitors. As described in the Examples the administration of glutamine or stabilized forms of glutamine significantly enhance the uptake of orally administered anti HIV drugs.

In one embodiment the glutamine containing compositions are administered prophylactically before and/or after chemotherapy or radiotherapy treatments or other therapeutic treatments to enhance the recovery of the intestinal barrier and improve the uptake of chemotherapeutic or other anticancer pharmaceuticals. In accordance with the present invention a method is provided for enhancing tissue repair and drug uptake after injury caused by infection, chemotherapy, radiotherapy, mechanical trauma, chemical stress, and thermal injury. The method comprises the steps of providing a patient suffering epithelial injury a composition comprising glutamine or stable glutamine derivatives in conjunction with a therapeutic agent to enhance uptake of the therapeutic agent. In one embodiment the stable glutamine derivative is alanyl-glutamine. The compositions of the present invention rapidly and significantly enhances cell migration, with a large improvement on the rapid epithelial repair after injury (named restitution). This cell migration occurs independently of DNA synthesis and cell proliferation The compounds used can be easily ingested.

In accordance with one embodiment the therapeutic agent and the glutamine-bearing compound are combined in a single composition that is administered orally to a mammal for treatment of a disease or condition. In one embodiment the composition comprises a glutamine-bearing compound and a pharmaceutical agent, wherein the glutamine-bearing compound is a peptide comprising an amino acid sequence selected from the group consisting of (GLN)ₙ, ALA-(GLN)ₙ, (ALA-GLN)ₙ, (GLN-Y-X)ₙ, (ALA-GLN-Y-X)ₙ, (Y—GLN-X)ₙ-[protease cleavage site]-(Y-GLN-X)ₚ and [(ALA-GLN)ₙ-protease cleavage site-(ALA-GLN)ₚ]ₘ wherein X and Y are independently GLN or ALA, n and p are integers independently selected from a range of 1 to 10, and m is an integer ranging from 1 to 20. In one embodiment the glutamine-bearing compound is a peptide selected from the group consisting of (ALA-GLN)ₙ or [(ALA-GLN)ₙ-protease cleavage site-(ALA-GLN)ₚ]ₘ wherein n and p are integers independently selected from a range of 1 to 3, and m is an integer ranging from 1 to 5. In one embodiment the glutamine-bearing compound is selected from the group consisting of alanyl-glutamine, alanyl-glutaminyl glutamine and gamma-glutamyl glutamine.

In accordance with one embodiment the composition comprises a glutamine-bearing compound selected from the group consisting of alanyl-glutamine, alanyl-glutaminyl glutamine and gamma-glutamyl glutamine, an antiretroviral drug and a pharmaceutically acceptable carrier. The antiretroviral drug can be selected from any of the known protease inhibitors and reverse transcriptase inhibitors, including but not limited to zidovudine, lamivudine, stavudine and didanosine, efavirenz, nevirapine and nelfinavir. More particularly, in one embodiment a method is provided for enhancing the absorption of antiretroviral drugs in HIV patients, including both prophylatic administration and more critically to those with AIDs who are suffering from diarrhea and wasting. The method comprises the steps of orally administering to the patient a glutamine-bearing composition in conjunction with a desired therapeutic drug, wherein the glutamine-bearing compound is a peptide comprises an amino acid sequence selected from the group consisting of (GLN)ₙ, ALA(GLN)ₙ and (ALA-GLN)ₙ wherein n is 1-20 In one embodiment the peptide sequence is an oligopeptide having from 2 to 5 amino acid units and selected from the group consisting of alanyl-glutamine, and alanyl-glutaminyl glutamine. In one embodiment the therapeutic drug is DDI or 3TC. The drug can be administered simultaneously with the glutamine-bearing composition or it can be administered before or after the administration of the glutamine-bearing composition (preferably within one hour after administration of glutamine containing peptide).

In one embodiment a pharmaceutical composition is provided for enhancing the uptake of pharmaceutical compounds by patients having damaged epithelial tissues. The composition comprises a glutamine derivative, a pharmaceutical agent and a pharmaceutically acceptable carrier. Preferably the glutamine derivative is a peptide sequence selected from the group consisting of ALA(GLN)ₙ and (ALA-GLN)ₙ wherein n is 1-5 and the pharmaceutical agent is selected from the group consisting of nutrients, antiviral agents and antibiotics. In another embodiment a method for enhancing the absorption of a drug administered to a patient having damaged epithelial tissues comprises the steps of orally administering to a patient a composition comprising a glutamine derivative and the drug, wherein the glutamine derivative is a peptide sequence selected from the group consisting of (GLN)ₙ, ALA(GLN)ₙ and (ALA-GLN)ₙ wherein n is 1-20. In one embodiment the drug is selected from the group consisting of antiviral agents and antibiotics.

In another embodiment, compositions comprising a pharmaceutical agent and a glutamine-bearing compound can be formulated as topical creams or ointments and administered topically to improve epithelial recovery in skin and oral mucosal lesions induced by chemotherapy, radiotherapy, mechanical trauma, chemical stress, and thermal injury. Considerable results can be obtained even with small concentrations of glutamine-bearing compounds. Advantageously, the glutamine-bearing compounds can be synthesized economically either chemically or biologically.

The present invention provides a simple general method for enhancing rapid epithelial repair an enhanced drug uptake in patients having deficient intestinal function, using compounds that can be easily ingested. The decreased intestinal function may be a directed result of the disease to be treated, may arise as a result of treatment or may be entirely unrelated to the treatment as a result of a physical injury or age. For example, this treatment can substantially reduce the incidence of secondary infection and inflammation, a major problem with patients being treated with chemotherapy or radiotherapy. Alternatively this treatment can be used in cases of disrupting infections, such as antibiotic associated diarrhea (e.g. Clostridium difficile diarrhea). Similar advantages may be expected with repair of any tissue injury, especially in malnourished, stressed (as by illness, injury or surgery), or elderly individuals. This process occurs independently of DNA synthesis and cell proliferation (McCormack et al., Am J Physiol 1992 263: G426-G435).

In accordance with one embodiment the composition is administered orally in the form of a liquid solution, liquid emulsion, powder, tablet, lozenge or capsule in a dosage range of from 0.05 to 1.0 g/kg (glutamine derivative) of said subject's body weight per day. The glutamine or stabilized glutamine compositions should be administered at a concentration of at least 30 g/day for an adult. Both glutamine and alanyl-glutamine are effective for this method, but alanyl-glutamine not only outperforms glutamine in enhancing dug uptake by intestinal tissue, but also is stable and can be kept in solution for a longer shelf-life.

Having documented the malabsorption of drugs in patients with chronic diarrhea (such as AIDS patients, for example) one aspect of the present invention is directed to the use of stable glutamine derivatives, such as alanyl-glutamine, to enhance the individual's ability to absorb orally administered drugs. In addition to the malabsorption of anti-retrovirals associated with AIDS patients that have chronic diarrhea, the use of glutamine derivatives to enhance the uptake of drugs is also relevant to patients receiving cancer chemotherapy, and patients suffering from enteric infections and malnutrition that damage the gut. In one embodiment, the glutamine derivative can be administered in conjunction with standard anti-tuberculosis drugs or antibiotics.

Great concern exists regarding the generation of drug resistant organisms (such as multiple drug resistant microbes, bacteria and viruses) when drug absorption is poor and thus systemic drugs levels are inferior. Administration of the glutamine-bearing compounds of the present invention in conjunction with standard antibiotic or antiviral therapies will enhance the absorption, and thus the effectiveness of the drug and minimize the cost of the therapy in individuals with compromised intestinal mucosa. Furthermore, the enhanced uptake of antiviral drugs will assist in providing optimal systemic drug levels leading to reduced incidence of drug resistance. Accordingly, one aspect of the present invention is to provide a method of reducing the emergence of antiretroviral drug resistance in patients with compromised intestinal absorptive function, such as in chronic wasting patients receiving orally administered antiretroviral therapy. The method comprises the step of orally administering to said patient a composition comprising a glutamine-bearing compound in conjunction with administration of the antiviral therapy. In one embodiment the antiretroviral drug is selected from the group consisting of protease inhibitors and reverse transcriptase inhibitors, and more particularly is selected from the group consisting of zidovudine, lamivudine, stavudine and didanosine, efavirenz, nevirapine and nelfinavir. In one embodiment the glutamine-bearing compound is a peptide comprising an amino acid sequence of the general formula (GLN)ₙ, (ALA-GLN)ₙ or [(ALA-GLN)ₙ-protease cleavage site-(ALA-GLN)ₚ]ₘ wherein n and p are integers independently selected from a range of 1 to 20, and m is an integer ranging from 1 to 5. In another embodiment the glutamine-bearing compound is a stabilized glutamine derivative peptide comprising an amino acid sequence of the general formula (ALA-GLN)ₙ, ALA(GLN)ₙ or [(ALA-GLN)ₙ-protease cleavage site-(ALA-GLN)ₚ]ₘ wherein n and p are integers independently selected from a range of 1 to 3, and m is an integer ranging from 1 to 5.

The pharmaceutical agent used in conjunction with the present invention can be any composition that is administered orally for absorption through the small intestine, and in particular include pharmaceuticals that are administered to treat or alleviate the symptoms associated with the condition that caused the intestinal tissue damage. For example, the pharmaceutical agent can be selected from the group consisting of nutrients, antiviral agents and antibiotics to treat or alleviate symptoms associated with malnutrition, AIDS or tuberculosis. In accordance with one embodiment the compositions of the present invention are administered orally in the form of a liquid solution, liquid emulsion, powder, tablet, lozenge or capsule in a dosage range of from 0.05 to 1.0 g/kg (of the glutamine derivative) of said subjects body weight per day.

The present glutamine derivatives can also be used to enhance the effectiveness and absorption of orally administered vaccine compositions. Accordingly, one aspect of the present invention relates to the administration of an antigenic composition (vaccine) in an oral dosage form, in conjunction with a glutamine derivative such as alanyl-glutamine or alanyl-glutaminyl glutamine. The glutamine derivative can be administered simultaneously with the vaccine or the vaccine can be administered after oral administration of the glutamine derivative. In one embodiment, an improved vaccine composition comprises a glutamine derivative, an antigen and a pharmaceutically acceptable carrier. The composition is administered orally to enhance the absorption and the effectiveness of the vaccine.

### EXAMPLE 1

In 1992, McCormack et al. established a model to study the process of cell migration involved in the early restitution of mucosal erosion. They used the small intestinal epithelial crypt cell line IEC-6 to show that cell migration occurs after wounding of a confluent layer of IEC-6 cells, and independently of DNA synthesis. This model system is used in the present invention in order to show the important effect of stable glutamine derivatives on intestinal epithelial restitution.

To investigate the ability of glutamine and stabilized glutamine derivatives to enhance the healing of mucosal tissues, a model system based on the migration of small intestinal epithelial crypt cells (line IEC-6) was used. The small epithelial crypt cell line (IEC-6 cells) was obtained from the American Type Culture Collection (Rockville, Md.). The medium consisted of Dullbecco's modified Eagle medium (DMEM) with 5% heat-inactivated FBS and 10 μg insulin and 50 μg gentamicin sulfate per milliliter. For the experiments, the cells were taken up with 0.25% trypsin plus 2.65 mM EDTA and plated at 6.25×10⁴ cell/cm² in 35 mm dishes thinly coated with Matrigel according to the manufacturer's instructions. The cells were fed every other day. To initiate migration, the cell layer was scratched with a single razor blade cut to approximately 27 mm in length. The scratch began at the diameter of the dish and extended over an area 7 to 10 mm wide. After the scratch, the cells were washed twice with sterile PBS. Then, medium containing no supplemental substances or medium containing glutamine or alanyl-glutamine (2.5 mmol/liter) was replaced, and the dishes were returned to the incubator. The experiments were carried out in triplicate. After 6 hours, 8 hours, and 24 hours, the dishes were removed from the incubator, and photographed using an inverted phase contrast microscope attached with camera The migration cells in five contiguous 0.1-mm squares were counted at ×100 magnification beginning at the scratch line and extending as far out as the cells had migrated. The counting was done in the area of the highest migration rate by means of an eyepiece reticle.

The experiments showed that Ala-Gln significantly enhanced cell migration compared to control 6 hours, 8 hours, and 24 hours after the scratch (for instance, alanyl-glutamine increased 107% and 173% related to control after 8 hours and 24 hours, respectively). Additionally, Ala-Gln was much better than glutamine (46% and 55% more) on enhancing cell migration after 6 and 8 hours.

### EXAMPLE 2

Plasma Levels of Antiretroviral Medications During Prolonged HIV-Associated Diarrhea

Advanced HIV infection is frequently complicated by diarrhea, disruption of bowel structure and function, and malnutrition. Applicants hypothesize that under such circumstances malabsorption of antiretroviral agents may occur, leading to subtheraputic drug dosing and treatment failure for individual patients. The resulting subtheraputic drug dosing may also facilitate development of resistant strains of HIV. Documentation that malabsorption does exist would have important consequences for the proper use of antiretroviral medications: dose adjustment and/or dietary adjuncts may be needed during periods of diarrheal illness. As described herein the peak levels of antiretroviral medications were compared in patients already started on medication by their physician. After informed consent blood samples were drawn from patients hospitalized for diarrhea or AIDS-associated wasting and antiretroviral medication levels were compared to levels obtained from patients well enough to not require hospitalization or meet criteria for severe diarrhea.

Study Site

The Sao Jose Hospital in Fortaleza, Brazil serves an HIV-infected population in an area of hyperendemicity for Cryptosporidium and other intestinal pathogens. The patient population is expected to be a heterogeneous group with respect to underlying cause of diarrhea, but with a very high prevalence of cryptosporidial infection, especially during the rainy season. Also represented in inpatients with diarrhea in Fortaleza are microsporidial infection, giardiasis, enterotoxigenic E. coli, and cholera.

Subjects

Eligible for this study are inpatient volunteers presently receiving drug treatment chosen by their physician for AIDS. A history of a positive serological test for HIV together with a history of at least one AIDS-defining illness will define a patient population at high risk for HIV-associated diarrhea and wasting. Inpatient subjects must have had diarrhea [defined as three or more stools daily of decreased consistency] for eight of the ten days prior to enrollment or intermittent diarrhea for two weeks over the two months prior to entry together with a weight loss of greater than 10% below baseline. Outpatient subjects will be recruited through their physicians, and will have a definition of AIDS as above, but do not meet the criteria for diarrhea or wasting and have an overall health status which has not required hospitalization in the last 2 months. Etiology of diarrhea will not be entry criterion, though this information will be collected as possible for future use in subgroup analysis. Exclusion criteria are (1) women who are pregnant, nursing, or not practicing effective contraception, (2) inability to give informed consent, and (3) evidence of severe hepatic disease or renal failure.

Drug Level Sampling

Peak drug levels will be determined by blood sampling two hours after the patient takes their antiretroviral medications as prescribed by their regular physician. Samples will be prepared in the laboratory of Dr. Lima for future analysis at a commercial laboratory.

Statistical Analysis

Comparison of peak plasma levels from subjects meeting the above diarrhea criteria will be compared with patients that do not. Levels of antiviral and antimycobacterial medications will be compared with Student's t-test for each antiretroviral medication tested.

### EXAMPLE 3

Use of Oral Glutamine Supplementation to Increase Absorption of Antiretroviral Medications in Patients Experiencing Prolonged HIV-Associated Diarrhea

Diarrhea and the AIDS wasting syndrome are frequent in individuals living in impoverished areas. The following study conducted by applicants in Northeast Brazil is the first to document the effects of glutamine and alanyl-glutamine on diarrhea and antiretroviral (ARV) drug absorption in patients with AIDS, diarrhea and wasting.

Materials and Methods:

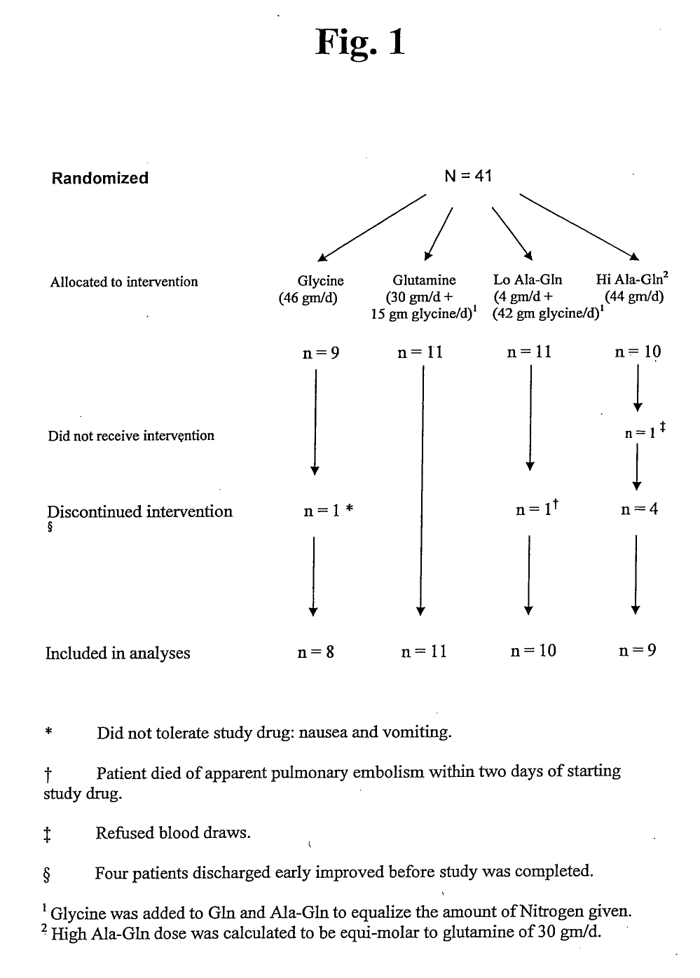

HUV positive patients on therapy presenting at the Hospital Sao Jose with diarrhea (≧3 stools/day×14 days) and wasting (loss of ≧10% of their body weight) from March 2001 to April 2002, were invited to participate. After informed consent, patients were assigned to one of four equi-nitrogenous blinded study groups including glycine controls (FIG. 1). Group A received 46 gm of oral glycine/day (Spectrum, Gardena, Calif.). Group B received 30 gm of oral glutamine/day (Cambridge Neutraceuticals, Cambridge, Mass.) and 15 gm of glycine, Group C received 4 grams alanyl-glutamine (Degussa/Creanova, Parsippany, N.J.) and 42 gm of glycine and Group D received 44 gm of alanyl-glutamine (Ala-Gln). The 44 gm alanyl-glutamine dose was calculated to contain equimolar glutamine to 30 gm of glutamine, and glycine was added to glutamine and low dose alanyl-glutamine groups to equalize the amount of nitrogen received by all participants. After ≧2 days of observed antiretroviral (ARV) drug therapy and again after 7 days of study drug, blood was drawn for 2-hour drug levels and viral genotyping and intestinal permeability studies were done. Control patients who were also on ARV medications but did not have diarrhea or wasting had 2 hour blood ARV drug levels taken.

Patients were asked daily about the frequency and consistency of their diarrhea and other gastrointestinal complaints including anorexia, vomiting, and abdominal cramping. Changes in frequency of diarrhea was scored as 0 if the total number of stools in the second half of the 9-day study was less than 2 fewer (or greater) than the number in the first half of the study (including the 2 days before study treatment was started); scored as − or +1 if stool frequencies were reduced or increased by 2 to 3, respectively; and − or +2 if the frequencies were reduced or increased by >3, respectively. In addition, consistencies were graded as liquid, soft or formed and scored as 0, 1 or 2 respectively and changes were added to the frequency scores above. Intestinal permeability was measured by urinary excretion of lactulose, mannitol and their ratio as previously described (Lima et al., Am J Gastroenterol 1997; 92(10):1861-1866). Lactulose and mannitol in urine was determined by high performance liquid chromatography (HPLC) and at Great Smokies Diagnostic Laboratory, Asheville, N.C.

Antiretroviral drug levels were determined at Tibotec-Virco, Mechelen, Belgium. Peak values were determined for each drug, and drug level ranges supplied by the manufacturer were used to define low levels. Viral genotype was determined by DNA sequencing at Visible Genetics, Suwanee, Ga. All genotypes were grouped with lade B subtypes in RT and protease regions. Two-tailed statistical analyses for comparison of paired values included Student's t-test and ANOVA and expressed as mean5 the standard deviation. Chi-square test or Fisher Exact test were used to compare proportions.

Results:

Forty-one patients were enrolled (age 23 to 52 years, median of 36 years; 29% female) and randomized as shown in Table 1 and FIG. 1.

TABLE 1
BASELINE CHARACTERISTICS OF ENROLLED PATIENTS BY
RANDOMIZED GROUP

| | ALL | GROUP A | GROUP B | GROUP C | GROUP D |
|---|---|---|---|---|---|
| Age (mean ± SD) | 36 ± 6 | 36 ± 6 | 38 ± 6 | 36.6 ± 6 | 36.9 ± 3 |
| Female/Male | 11/30 | 1/8 | 6/6 | 3/8 | 1/8 |
| Weight (kg) | 47.9 ± 9.54 | 46 ± 9.23 | 54 ± 11.4 | 44 ± 7.88 | 46 ± 7.5 |
| BMI | 17 | 16 | 21.1 | 16.4 | 14.3 |

Glutamine levels (μmol/L ± SD)

| | | | | | |
|---|---|---|---|---|---|
| Before | 23.16 ± 10.4 | 23.04 ± 11.48 | 20.53 ± 9.91 | 24.16 ± 12.07 | 23.96 ± 9.23 |
| treatment | | | | | |
| After | 23.29 ± 12.61 | 22.36 ± 10.13 | 21.83 ± 12.84 | 20.88 ± 14.93 | 26.12 ± 7.59 |
| treatment | | | | | |
| Mean change | 0.13 | −0.68 | 1.29 | −3.29 | 2.17 |

Stool Results

| | | | | | |
|---|---|---|---|---|---|
| Lactoferrin | 24 (60%) | 6 | 8 | 3 | 7 |
| Cryptosporidium | 4 (10%) | 1 | 3 | | |
| S. stercoralis | 3 | 1 | 2 | | |
| T. trichura | 1 | | 1 | | |
| I. belli | 1 | | | 1 | |
| Plasma HIV RNA | | 101,000 | 485,000 | 400,000 | 270,000 |
| CD4 cells | | 97 | 123 | 86 | 84 |
| (Range) | | (19-386) | (11-506) | (19-183) | (3-285) |

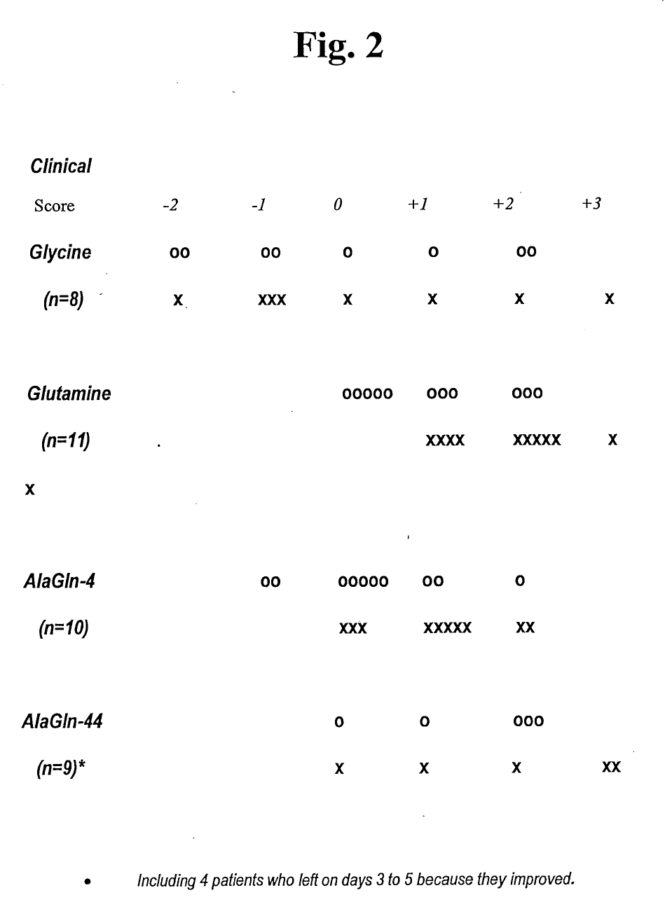

Clinical responses among the 38 evaluable patients are summarized in FIG. 2. Clinical response are indicated by diarrhea frequency (o) and consistency (with frequency, x) for seven days, scored as described in materials and methods. Eight of 9 (89%) in the high dose Ala-Gln group improved (including the 4 patients who left study on day 3 because they improved) vs. 3 of 8 (38%) for the control patients (p<0.05). Twenty-six of 30 (87%) taking any study drug improved (p<0.01 vs. glycine controls), while 19 of 20 (95%) taking full dose Gln or Ala-Gln improved (p<0.003 vs. glycine controls).

Antiretroviral drug levels were determined on all 33 study patients with diarrhea and wasting who had specimens available. Regimens included zidovudine (AZT) and lamivudine (3TC) or stavudine (D4T) and didanosine (ddI) with either a non-nucleoside reverse transcriptase inhibitor (NNRTI, either efavirenz or nevirapine) or a protease inhibitor (PI, in most cases nelfinavir). Compared with controls without diarrhea or wasting, study patients had significant ARV drug malabsorption of ddI [mean 397±151 ng/mL (n=12) vs. 737±226 ng/ml (n=3); p<0.007] and 3TC [mean 1503 ng/ml (n=18) vs 2813 ng/ml (n=9); p=0.02]. All patients on ddI, 3TC, ritonavir, saquinavir, and 12/14 (85%) of patients on efavirenz, had drug levels below the expected range. Four of 9 (44%) patients on nelfinavir and 3/15 (20%) patients on stavudine also had low drug levels.

Improved Antiretroviral Absorption with Glutamine/Alanyl-Glutamine

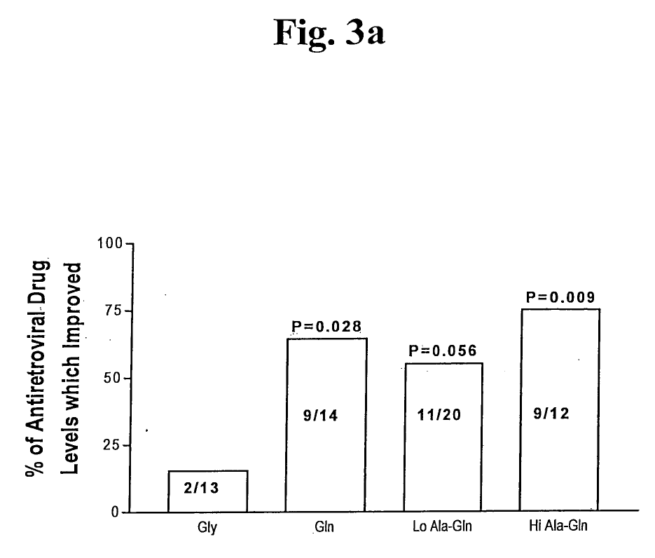

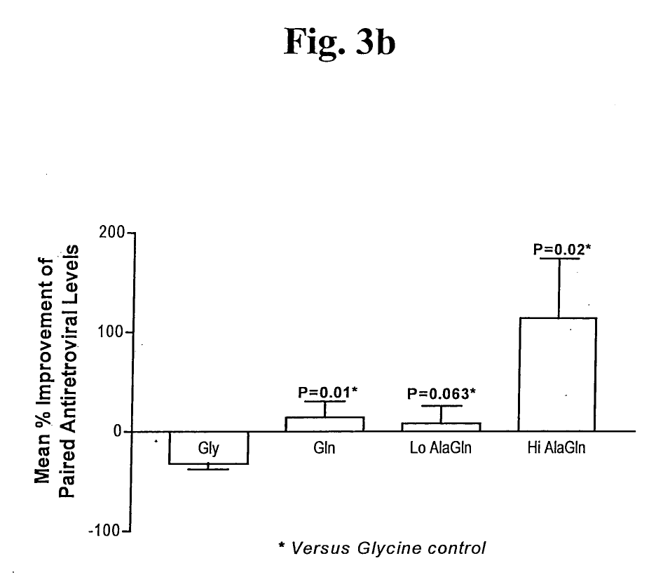

Nine of 12 (75%) of peak paired Nucleoside Reverse Transcriptase Inhibitor (NRTI) levels improved in patients given high dose Ala-Gln (p=0.009); 9/14 (64.3%) of drug levels improved in the Gln (p=0.028) group and 11/20 (55%) drug levels improved in the low dose Ala-Gln group (p=0.056) vs. 2/13 (15.4%) of glycine controls (FIG. 3a). In patients receiving Gln or either dose of Ala-Gln, 29/46 (63%) of paired peak drug levels improved (p=0.006 vs. glycine controls). Similar significant improvements with high dose Ala-Gln (p=0.02) and Gln (p=0.03) were seen when all ARV drugs (including 17 NNRTI and 14 PI levels) were combined. Shown in FIG. 3b are the mean percent improvement in antiretroviral drug levels (greatest with high dose Ala-Gln 113%, p=0.02; then Gln 14%, p=0.01; then low dose Ala-Gln 8%, p=0.06 compared with glycine controls (−32%)). Taken together, all 3 treatment groups had a mean increase in drug levels of 45% (p=0.02 vs. glycine controls).

Eighteen patients were on 3TC, 10 patients improved with therapy, 6 on Ala-Gln (4 low dose and 2 high dose), 2 on Gln and 2 on glycine. Of the 8 patients whose 3TC levels did not improve, 3 were on low dose Ala-Gln, 3 were on glycine, and 2 were on Gln; none of the 8 who did not improve their 3TC levels were in the high dose Ala-Gln group. Fifteen patients were on D4T and seven had an increase in their levels; 6 were receiving Ala-Gln (3 high dose and 3 low dose) and the other received Gln. Of the 8 who did not increase their D4T levels, 3 were receiving Gln, 2 low dose Ala-Gln, 1 high dose Ala-Gln and 2 were on glycine. In patients taking AZT (n=14), six had improved levels with therapy and all were in the Gln and Ala-Gln group (p=0.07). Of the eight that did not improve half were in the control group while 3 were taking low dose Ala-Gln and 1 was on high dose Ala-Gln.

HIV genotyping from 9/16 patients showed 65 resistance mutations. Forty-five of these 65 mutations (69.2%) were associated with sub-therapeutic levels of a drug to which resistance is conferred. Four patients had viruses with mutations conferring resistance to all 13 drugs being given (46.2% being malabsorbed); 3 patients had viruses with mutations conferring resistance to 2 of 3 drugs being given (6 being malabsorbed, 4 of which were among the 6 resistance mutations); one patient had virus with mutations conferring resistance to 3 of 4 drugs being given (1 being malabsorbed) and one patient had virus with mutations conferring resistance to 1 of 3 drugs. These 9 patients were taking a total of 29 antiretroviral drugs; 16 (55%) were being malabsorbed and 11 (69%) of these were associated with a resistance genotype in these patients. Overall, of the 50 antiretroviral drugs levels in 16 patients whose HIV genotypes were determined, 26 (52%) demonstrated low drug levels and 11 (42%) of these were associated with a resistance genotype.

The most common mutations seen were at codon 184 conferring resistance to 3TC in all cases. Other NRTI-associated mutations (NAMs) found were M41L, D67N, K70R and T215F. Multi-NRTI resistance with codon 151 mutation complex was noted in two patients and one patient had 69 insertion complex. Overall, 6/7 patients who were on AZT and 3TC had a resistant virus to one or more drug they were on; 4/6 patients taking a NNRTI had a mutation at codon 103; one patient taking a PI had a major mutation at codon 82 and several minor mutations.

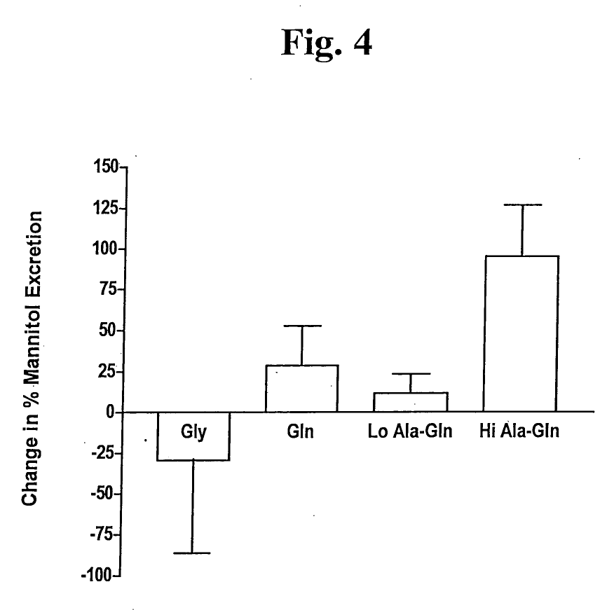

Intestinal permeability studies show graded trends for the percent mannitol excretion that matched the clinical and drug absorption responses by treatment groups (FIG. 4). Percent mannitol excretion in all treatment groups improved 25.4% (vs. glycine controls; p=0.071). Lactulose absorption was not affected.

Discussion:

In Brazil, more than 60% of all patients with HIV present initially with diarrhea where significant malabsorption occurs. Our findings that oral glutamine or its stabler and more soluble derivative, alanyl-glutamine significantly improve diarrheal symptoms and ARV drug absorption. Thus the administration of glutamine containing peptides provides an important novel approach toward alleviating these symptoms and potentially toward improving ARV drug efficacy, especially in tropical, developing countries.

Tepper et. al. have described impaired mannitol absorption in patients with AIDS (Tepper et al., Am J Gastroenterol 1994; 89(6):878-882), and we have reported a 3-fold increase in lactulose: mannitol ratios, reflecting intestinal barrier disruption and malabsorption in HUV infected patients with diarrhea in comparison to HIV infected patients without diarrhea, and a 10-fold increase versus healthy controls, again predominantly due to mannitol absorption (Lima et al., Am J Gastroenterol 1997; 92(10):1861-1866). These results also suggest that even HIV infected patients without diarrhea could benefit from the co-administration of glutamine or stabilized glutamine derivatives along with antiviral therapy since there is a 7-fold increase in lactulose: mannitol ratios in HIV infected patients without diarrhea versus healthy controls.

Glutamine supplementation has been shown to improve intestinal mucosal structure and function following injury by chemotherapy, radiotherapy (Ziegler et al., Ann Intern Med 1992; 116(10):821-828) or prolonged parenteral nutrition Tremel et al., Gastroenterology 1994; 107(6):1595-1601) and, in addition, it is an effective oral rehydration as well as nutrition therapy (see U.S. Pat. No. 5,561,111). Consistent with this, significant clinical improvements in stool frequency and consistency were noted in the Gln or Ala-Gln (p<0.01 vs. glycine controls) groups, with the greatest improvements seen in the high dose Ala-Gln group (p<0.003, when combined with Gln).

Based on known increased intestinal permeability documented in HIV-positive patients with diarrhea (Tepper et al., Am J Gastroenterol 1994; 89(6):878-882 and Lima et al., Am J Gastroenterol 1997; 92(10):1861-1866), Noyer et. al., Am J Gastroenterol 1998; 93(6):972-975 have demonstrated low dose Gln (4-8 g/day, over 28 days) showed less worsening of intestinal permeability with the 4 g/day dose; and, at the 8 g/day dose, there was stabilization of intestinal permeability and improved absorption of mannitol (an indicator of intestinal absorptive surface area). The present study shows that at higher doses of Gln and equi-nitrogenous doses of Ala-Gln an increase in percent mannitol excretion was noted though it did not reach significance with only one week of supplementation. These trends suggest that, at least initially over the first week, the primary effect of Gln and Ala-Gln is on rebuilding absorptive surface area, rather than on changes in barrier function. Gln levels were significantly low in all patients (23.16±10.4 μmol/L; range 4.79-43.81 μmol/L; compared to the normal range of 500-700 μmol/L). This is analogous to studies that have shown the HIV positive patients with wasting have significantly lower levels of Gln (Young et al., J Am Diet Assn 1992; 92(suppl):A88).

The two-hour time point was used as a measure of absorption since this is the time when most ARV drugs peak in the plasma. However, it is possible that some patients could have had delayed absorption with their peak level occurring at 4 or 6 hours after dosing. Low levels of antiretroviral drugs occurred in all 33 patients with diarrhea and wasting, significantly so for ddI (p<0.007 vs. controls) and 3TC (p<0.02 vs controls), possibly driving viral resistance. Trends from this and other studies (Brantley et al., Braz J Infect Dis 7[1], 16-22. 2003) reveal that certain drugs are observed as being more prone to malabsorption than others. Although the high frequency of resistance mutations was not significantly greater for drugs being malabsorbed in this study, all patients had been on multiple regimens. Further longitudinal studies are therefore needed of patients not previously treated to determine whether ARV drug malabsorption leads to resistant virus and whether Gln of Ala-Gln based ORNT prevents emergence of ARV drug resistance.

Several studies have shown concentration managed therapy with NRTIs to be clinically feasible and associated with better outcome (31-33). We found that NRTI levels significantly improved after Gln or Ala-Gln based ORNT whether assessed as the percent of patients with improved levels or as the mean change in serum drug concentrations, both showed that again the greatest effects were seen with high dose Ala-Gln (75% of patients; mean 113% increase), and then Gln followed by low dose Ala-Gln. Whether Gln or Ala-Gln improves NNRTI or PI absorption requires further study.

In conclusion, significant improvements in symptoms and antiretroviral drug malabsorption (both percent of patients and mean change in serum levels) occurred with supplementation of Gln or its analogue Ala-Gln. Improvements in all clinical and laboratory parameters measured were greatest with 44 gm/d of Ala-Gln followed by 30 gm/d of Gln which was better than 4 gm/d of Ala-Gln, all better than 46 gm/day of glycine used as control. The dose relatedness and apparent superiority of alanyl-glutamine, in addition to its greater stability and solubility and superior ability to drive water and electrolyte absorption, demonstrate its promise in improving diarrhea and malabsorption and thus potentially improving ARV drug therapy and reducing the emergence of drug resistance in patients with HIV/AIDS in tropical, developing areas.

1. A method of enhancing the absorption of a pharmaceutical agent administered orally to a mammal, said method comprising the steps of
administering to said mammal a composition comprising a glutamine-bearing compound; and
administering orally to said mammal the pharmaceutical agent.

2. The method of claim 1 wherein the glutamine composition is administered prior to the administration of the pharmaceutical agent.

3. The method of claim 1 wherein the glutamine composition is administered simultaneously with the administration of the pharmaceutical agent.

4. The method of claims 2 or 3 wherein the glutamine composition is administered orally.

5. The method of claim 4 wherein the glutamine-bearing compound is glutamine, a polymer of glutamine, or a stabilized derivative of glutamine.

6. The method of claim 5 wherein the glutamine-bearing compound is linked via its amino or carboxy terminus to a secondary peptide or secondary protein.

7. The method of claim 5 wherein the glutamine-bearing compound comprises an amino acid sequence selected from the group consisting of (GLN)N (ALA-GLN)ₙ, (GLN-Y-X)ₙ, (ALA-GLN-Y-X)ₙ, (Y-GLN-X)ₙ-[protease cleavage site]-(Y-GLN-X)ₚ and [(ALA-GLN)ₙ-protease cleavage site-(ALA-GLN)ₚ]ₘ wherein X and Y are independently GLN or ALA, n and p are integers independently selected from a range of 1 to 100, and m is an integer ranging from 1 to 20.

8. The method of claim 7 wherein the glutamine-bearing compound is MET(ALA-GLN-GLN)ₙ, MET(ALA-GLN)ₙ or MET[(ALA-GLN)ₙ-protease cleavage site-(ALA-GLN)ₚ]ₘ wherein n and p are integers independently selected from a range of 1 to 10, and m is an integer ranging from 1 to 5.

9. The method of claim 5 wherein the stabilized glutamine derivative comprises an amino acid sequence of the general formula ALA-(GLN)ₙ, (ALA-GLN)ₙ or [(ALA-GLN)ₙ-protease cleavage site-(ALA-GLN)ₚ]ₘ wherein n and p are integers independently selected from a range of 1 to 100, and m is an integer ranging from 1 to 20.

10. The method of claim 5 wherein the glutamine-bearing compound is ALA-(GLN)ₙ or (ALA-GLN)_{q} wherein n is an integer ranging from 1 to 4, and q is an integer ranging from 1 to 3.

11. The method of claim 1 or 6 wherein the mammal is a human subject having compromised intestinal function.

12. The method of claim 11 wherein the human subject is HIV positive and the administered pharmaceutical agent is an antiretroviral drug.

13. A composition for enhancing the uptake of a pharmaceutical agent by a mammal, wherein the mammal is suffering from intestinal mucosa damage, said composition comprising a glutamine-bearing compound, or pharmaceutically-acceptable salt thereof, and a pharmaceutical agent.

14. The composition of claim 13 wherein the glutamine-bearing compound is glutamine, a polymer of glutamine, or a stabilized derivative of glutamine.

15. The composition of claim 14 wherein the glutamine-bearing compound is linked via its amino- or carboxy terminus to a secondary peptide or secondary protein.

16. The composition of claim 14 wherein the stabilized glutamine derivative comprises an amino acid sequence (ALA-GLN)ₙ or [(ALA-GLN)ₙ-protease cleavage site-(ALA-GLN)ₚ]ₘ wherein n and p are integers independently selected from a range of 1 to 100, and m is an integer ranging from 1 to 20.

17. The composition of claim 13 wherein the glutamine-bearing compound comprises an amino acid sequence selected from the group consisting of (GLN)N (ALA-GLN)ₙ, (GLN-Y-X)ₙ, (ALA-GLN-Y-X)ₙ, (Y-GLN-X)ₙ-[protease cleavage site]-(Y-GLN-X)ₚ and [(ALA-GLN)ₙ-protease cleavage site-(ALA-GLN)ₚ]ₘ wherein X and Y are independently GLN or ALA, n and p are integers independently selected from a range of 1 to 100, and m is an integer ranging from 1 to 20.

18. The method of claim 17 wherein the glutamine-bearing compound is MET(ALA-GLN-GLN)ₙ, MET(ALA-GLN)ₙ or MET[(ALA-GLN)ₙ-protease cleavage site-(ALA-GLN)ₚ]ₘ wherein n and p are integers independently selected from a range of 1 to 10, and m is an integer ranging from 1 to 5.

19. The method of claim 13 wherein the glutamine-bearing compound is ALA-(GLN)ₙ, or (ALA-GLN)_{q} wherein n is an integer ranging from 1 to 4, and q is an integer ranging from 1 to 3.

20. The composition of any of claims 13-19 wherein the therapeutic agent is an antiretroviral drug.

21. The composition of claim 20 wherein the antiretroviral drug is selected from the group consisting of protease inhibitors and reverse transcriptase inhibitors.

22. The composition of claim 21 wherein the antiretroviral drug is selected from the group consisting of zidovudine, lamivudine, stavudine and didanosine, efavirenz, nevirapine and nelfinavir.

23. The composition of claims 16, 17 or 18 wherein the protease cleavage site is selected from the group consisting of trypsin, chemotrypsin, Factor Xa and TEV.

24. A method of reducing the emergence of antiretroviral drug resistance in a chronic wasting patient receiving orally administered antiretroviral therapy, said method comprising the steps of
administering to said patient a composition comprising a glutamine-bearing compound; and
administering to said patient an antiretroviral drug.

25. The composition of claim 24 wherein the antiretroviral drug is selected from the group consisting of protease inhibitors and reverse transcriptase inhibitors.

26. The composition of claim 25 wherein the antiretroviral drug is selected from the group consisting of zidovudine, lamivudine, stavudine and didanosine, efavirenz, nevirapine and nelfinavir

27. The method of claims 24 wherein the glutamine composition is administered orally.

28. The method of claim 27 wherein the glutamine composition is administered prior to the administration of the pharmaceutical agent.

29. The method of claim 28 wherein the administration of the pharmaceutical agent is accompanied by a simultaneous administration of a second glutamine composition.

30. The method of claim 24 wherein the glutamine-bearing compound is comprises an amino acid sequence of the general formula (GLN)ₙ, (ALA-GLN-GLN)_{q}, or (ALA-GLN)_{q} wherein n is an integer ranging from 1 to 5 and q is an integer ranging from 1 to 3.
